(19) United States
(12) Patent Application Publication
(10) Pub. No.: US 2023/0405174 A1
(43) Pub. Date: Dec. 21, 2023

(54) BLOWER FAN HAVING STERILIZATION MODULE INTEGRATED THEREWITH

(71) Applicants: MEEKYUNGTEC CO., LTD, Gyeonggi-do (KR); JK ELECTRO CO., LTD., Seoul (KR)

(72) Inventors: Seung Zu YOUNG, Gyeonggi-do (KR); Se Been OH, Gyeonggi-do (KR); Hun Sang PARK, Incheon (KR)

(21) Appl. No.: 18/251,682

(22) PCT Filed: Dec. 23, 2020
(86) PCT No.: PCT/KR2020/018978

§ 371 Date: May 3, 2023

(30) Foreign Application Priority Data
Nov. 4, 2020 (KR) 10-2020-0146141

(51) Int. Cl. A61L 9/20; F04D 29/056; F04D 25/08

(52) U.S. Cl. CPC A61L 9/20; F04D 29/056; F04D 25/08; F04D 29/281

(57) ABSTRACT

A blower fan 100 having a sterilization module integrated therewith includes a stationary part 10 including a central shaft 12 formed at the center to protrude upward, a bracket 16 extending horizontally from the bottom of the central shaft, and a coil 18 disposed around the central shaft; a rotary part 20 rotating about the central shaft of the stationary part and including a blade 26 formed at the circumference and a permanent magnet 28 disposed around the coil of the stationary part at the inner side of the blade; a bearing 30 having an inner race 32 fixed to the stationary part 10 and an outer race 34 fixed to the rotary part 20; and a sterilization module arranged to diffusely radiate light upward from the top of the central shaft of the stationary part 10 and maintained in a stationary state when the rotary part 20 rotates.

### TECHNICAL FIELD

The present invention relates to a blower fan having a sterilization module integrated therewith, and more particularly, to a blower fan having a sterilization module integrated therewith, in which the sterilization module is installed on a stationary part of the blower fan, and the sterilization module is kept in a stationary state when a rotary part rotates.

### BACKGROUND ART

As the amount of various pollutants generated at industrial sites and the amount of automobile emissions increase and environmental pollution is becoming serious, the demand for a pleasant and favorable environment is increasing.

In particular, in recent years, a large amount of fine dust and yellow dust from China has been entering Korea, and a large amount of fine dust is generated in Korea by pollutants emitted by automobiles and industrial companies. As a result, the total amount of fine dust stagnating in Korea has increased, and the air quality has often deteriorated. Therefore, many people are taking measures to cope with air pollution. As a result, it has become almost a necessity to have an air purifier in the home to breathe in clean air. A vehicle air purification device, which purifies the air in the vehicle interior, is becoming an essential item.

In general, air purifiers in the related art utilize a structure that includes an air filter and blows air through a fan. While these air purifiers filter out inhaled bacteria (including mold) from the air, the air purifiers are inconvenient because the filters need to be changed regularly and frequently. In addition, because filters cannot kill germs, germs can live(grow) and multiply on the filter. When air conditioning is poor, the filters may actually release more polluted air than when not used.

Meanwhile, the blower device that is mounted on the air purifier and uses a fan is equipped with various sterilization devices to purify the air and remove microorganisms, but the blower device has a problem in that it is necessary to have a separate mounting space, and the cost increases. In addition, the blower device using the fan in the related art can kill the microorganisms contained in the air passing through the fan by using the separate sterilization device described above. However, there is a problem in that the microorganisms attached to the fan and the contamination of the fan cannot be eliminated.

To solve these problems, Korean Patent No. 2024556 discloses a sterilization fan structure 1 comprising an ultraviolet light-emitting diode in which the ultraviolet light-emitting diode is coupled to a fan blade, which may not only decontaminate the air passing through the fan, but also eliminate microorganisms adhering to and living in the fan and contamination of the fan (see FIG. 1).

However, in the sterilization fan structure of Korean Patent No. 2024556, an ultraviolet light-emitting diode 3 having a sterilization function is coupled to a blade 2 and rotates together with the blade. Therefore, in order to supply power to the rotating ultraviolet light-emitting diode 3, one end of a (+) wire 4 and one end of a (−) wire 5 need to be connected to the light-emitting diode 3, the other end of the (+) wire 4 and the other end of the (−) wire 5 need to be connected to (+)(−) electrode rods 6 and 7, and brushes 8 and 9 need to be connected to the (+)(−) electrode rods 6 and 7 (see FIG. 2).

As described above, Korean Patent No. 2024556 uses the electrode rods and the brushes to supply power to the ultraviolet light-emitting diode, which not only complicates the structure of the fan structure, but also degrades the efficiency of sterilization due to defects in contact between the electrode rods and the brushes, and requires regular replacement of the brushes.

### DISCLOSURE

### Technical Problem

The present invention has been made in an effort to solve the above-mentioned problem, and an object of the present invention is to provide a blower fan having a sterilization module integrated therewith, in which an electrode rod and a brush structure, which are configured to supply power to a sterilization module, are not used, which may improve efficiency of a sterilization operation of a blower fan, simplify a structure of the blower fan, and facilitate maintenance. However, the object of the present invention is illustrative, and the scope of the present invention is not limited by the object.

### Technical Solution

To achieve the above-mentioned object, an embodiment of the present invention provides a blower fan having a sterilization module integrated therewith, the blower fan including: a stationary part including a central shaft protruding upward from a center thereof, a bracket extending horizontally from a lower end of the central shaft, and a coil disposed around the central shaft; a rotary part including a blade formed on a peripheral portion thereof, and a permanent magnet disposed around coil of the stationary part at the inner side of the blade, the rotary part being configured to rotate about the central shaft of the stationary part; a bearing having an inner race fixed to the stationary part and an outer race fixed to the rotary part; and a sterilization module disposed at an upper end of the central shaft of the stationary part to diffusely radiate light upward, the sterilization module being configured to be maintained in a stationary state when the rotary part rotates.

In the blower fan having the sterilization module integrated therewith according to the embodiment of the present invention, the sterilization module may be configured as a UV-C LED.

In the blower fan having the sterilization module integrated therewith according to the embodiment of the present invention, the central shaft of the stationary part may include a first protruding portion extending from an upper surface of the bracket to a predetermined height and a connection part fixedly inserted into the first protruding portion and extending to an upper surface of the rotary part; the inner race of the bearing may be fixed to an outer peripheral surface of the connection part of the stationary part the outer race may be fixed to an inner peripheral surface of a rotary shaft of the rotary part; and the sterilization module may be disposed at the upper end of the connection part of the stationary part to diffusely radiate light upward and maintained in the stationary state when the rotary part rotates.

In the blower fan having the sterilization module integrated therewith according to the embodiment of the present invention, the central shaft of the stationary part may include a first protruding portion extending from an upper surface of the bracket to a predetermined height and a connection part fixedly inserted into the first protruding portion and having a flange portion provided at an upper end thereof and extending horizontally; the inner race of the bearing may be fixed to a peripheral portion of the flange portion of the connection part of the stationary part and the outer race may be fixed to an inner peripheral surface of a rotary shaft of the rotary part; and the sterilization module may be disposed on the flange portion of the connection part of the stationary part to diffusely radiate light upward and maintained in the stationary state when the rotary part rotates.

In the blower fan having the sterilization module integrated therewith according to the embodiment of the present invention, the stationary part may further include a second protruding portion disposed outside a first protruding portion and extending from an upper surface of the bracket to a predetermined height concentrically with the first protruding portion; and the inner race of the bearing may be fixed to an outer peripheral surface of the second protruding portion of the stationary part and the outer race may be fixed to an inner peripheral surface of a rotary shaft of the rotary part.

In the blower fan having the sterilization module integrated therewith according to the embodiment of the present invention, the bearing may be a ball bearing.

According to an embodiment of the present invention, a vehicle air purification device may include any one of the above-mentioned blower fans having the sterilization module integrated therewith.

In the vehicle air purification device according to the embodiment of the present invention, the blower fan having the sterilization module integrated therewith may be disposed rearward of a filter unit.

### Advantageous Effects

According to the embodiment of the present invention configured as described above, the electrode rod and the brush structure, which are configured to supply power to the sterilization module, are not used in the blower fan having the sterilization module integrated therewith, which may improve the efficiency of the sterilization operation of the blower fan, simplify the structure of the blower fan, and facilitate maintenance. Of course, the scope of the present invention is not limited by the effect.

### BRIEF DESCRIPTION OF DRAWINGS

FIG. 1 is a schematic use state view of a sterilization fan structure including an ultraviolet light-emitting diode in the related art.

FIG. 2 is a configuration view schematically illustrating a wire connection structure of the ultraviolet light-emitting diode of the sterilization fan structure in FIG. 1.

FIG. 3 is a schematic view illustrating a state in which ultraviolet rays are radiated toward a filter unit from a sterilization module of a vehicle air purification device to which a blower fan having a sterilization module integrated therewith according to an embodiment of the present invention is applied.

FIG. 4 is a schematic view illustrating a state in which ultraviolet rays are radiated upward radially from the sterilization module of the vehicle air purification device to which the blower fan having the sterilization module integrated therewith according to the embodiment of the present invention is applied.

FIG. 5 is a top plan view of the vehicle air purification device to which the blower fan having the sterilization module integrated therewith according to the embodiment of the present invention is applied.

FIG. 6 is a side view of the vehicle air purification device to which the blower fan having the sterilization module integrated therewith according to the embodiment of the present invention is applied.

FIG. 7 is a bottom plan view of the vehicle air purification device to which the blower fan having the sterilization module integrated therewith according to the embodiment of the present invention is applied.

FIG. 8 is a cross-sectional view of a blower fan in which a rotary shaft of a rotary part and a bearing are disposed inside a coil of a stationary part of a blower fan having a sterilization module integrated therewith according to a first embodiment of the present invention.

FIG. 9 is a cross-sectional view of a blower fan in which a rotary shaft of a rotary part and a bearing are disposed inside a permanent magnet of the rotary part in a blower fan having a sterilization module integrated therewith according to a second embodiment of the present invention.

FIG. 10 is a cross-sectional view of a blower fan in which a rotary shaft of a rotary part and a bearing are disposed outside a permanent magnet of the rotary part in a blower fan having a sterilization module integrated therewith according to a third embodiment of the present invention.

### MODE FOR INVENTION

The objects, features, and advantages of the present invention will become more apparent with reference to the following embodiments associated with the accompanying drawings.

Specific structural or functional descriptions of embodiments of the present invention are exemplified only for the purpose of explaining the embodiments according to the concept of the present invention, the embodiments according to the concept of the present invention may be carried out in various forms, and it should not be interpreted that the present invention is limited to the embodiments described in this specification or application.

Because the embodiments according to the concept of the present invention may be variously changed and may have various forms, specific embodiments will be illustrated in the drawings and described in detail in the present specification or application. However, the descriptions of the specific embodiments are not intended to limit embodiments according to the concept of the present invention to the specific embodiments, but it should be understood that the present invention covers all modifications, equivalents and alternatives falling within the spirit and technical scope of the present invention.

The terms such as “first” and/or “second” may be used to describe various constituent elements, but these constituent elements should not be limited by these terms. These terms are used only for the purpose of distinguishing one constituent element from other constituent elements. For example, without departing from the scope according to the concept of the present invention, the first constituent element may be referred to as the second constituent element, and similarly, the second constituent element may also be referred to as the first constituent element.

When one constituent element is described as being “connected” or “coupled” to another constituent element, it should be understood that one constituent element can be connected or coupled directly to another constituent element, and an intervening constituent element can also be present between the constituent elements. When one constituent element is described as being “connected directly to” or “coupled directly to” another constituent element, it should be understood that no intervening constituent element is present between the constituent elements. Other expressions, that is, “between” and “just between” or “adjacent to” and “directly adjacent to”, for explaining a relationship between constituent elements, should be interpreted in a similar manner.

The terms used in the present specification are used only for the purpose of describing particular embodiments and are not intended to limit the present invention. Singular expressions include plural expressions unless clearly described as different meanings in the context. In the present application, it will be appreciated that terms “including” and “having” are intended to designate the existence of characteristics, numbers, steps, operations, constituent elements, and components described herein or a combination thereof, and do not exclude a possibility of the existence or addition of one or more other characteristics, numbers, steps, operations, constituent elements, and components, or a combination thereof in advance.

Unless otherwise defined, all terms used herein have the same meaning as commonly understood by those skilled in the art to which the present invention pertains. The terms such as those defined in a commonly used dictionary should be interpreted as having meanings consistent with meanings in the context of related technologies and should not be interpreted as ideal or excessively formal meanings unless explicitly defined in the present specification.

Hereinafter, the present invention will be described in detail by describing the embodiments of the present invention with reference to the accompanying drawings. Like reference numerals indicated in the respective drawings refer to like members.

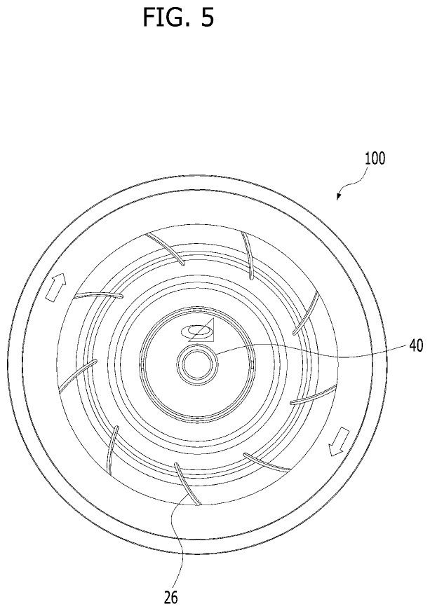

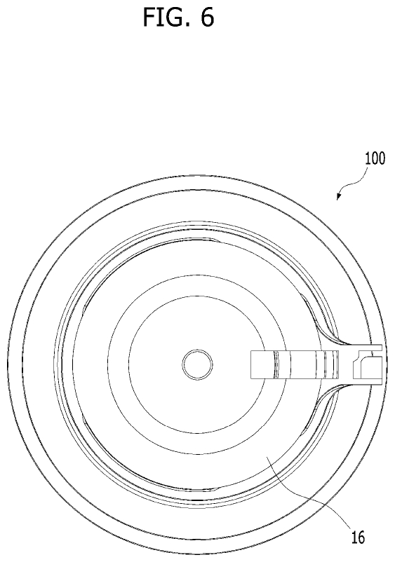

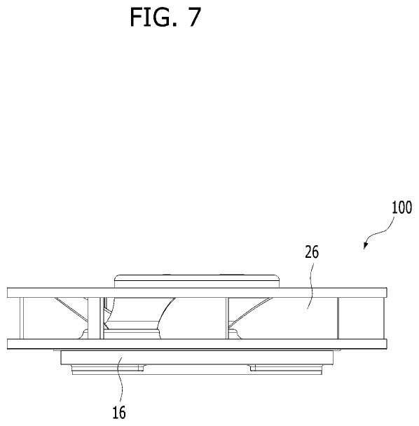

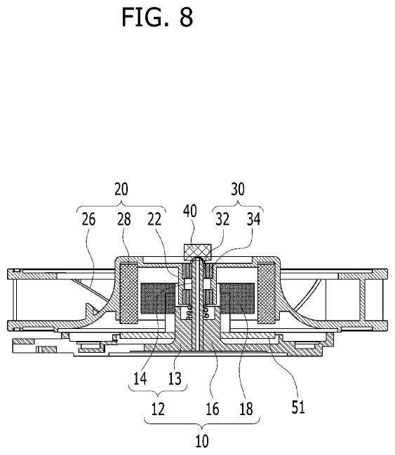

FIG. 5 is a top plan view of a vehicle air purification device to which a blower fan having a sterilization module integrated therewith according to an embodiment of the present invention is applied, FIG. 6 is a side view of the vehicle air purification device to which the blower fan having the sterilization module integrated therewith according to the embodiment of the present invention is applied, FIG. 7 is a bottom plan view of the vehicle air purification device to which the blower fan having the sterilization module integrated therewith according to the embodiment of the present invention is applied, and FIG. 8 is a cross-sectional view of a blower fan in which a rotary shaft of a rotary part and a bearing are disposed inside a coil of a stationary part of a blower fan having a sterilization module integrated therewith according to a first embodiment of the present invention.

In general, sterilizing a flowing gas takes more time than sterilizing a stationary gas, which makes it more difficult to sterilize the gas. Therefore, it is known that it is difficult to directly sterilize the air passing through a blower fan. Therefore, in an air purifier, it is more effective to sanitize the filter directly than to sterilize the air passing through the filter that filters out bacteria, viruses, and the like.

In general, air flows in the order of air inlet→filter unit→blower fan→air outlet in an air purifier used indoors or a vehicle air purification device. Therefore, to directly sterilize the filter, it may be considered to install a sterilization device between the air inlet and the filter unit or between the filter unit and the blower fan. However, in case that the sterilizer is disposed as described above in the air purifier or the vehicle air purification device, there is a risk that the sterilizer may obstruct the air flow and reduce the efficiency of the air purifier or the vehicle air purification device.

To prevent this problem from occurring, it may be considered to attach the sterilizer directly to the blower fan.

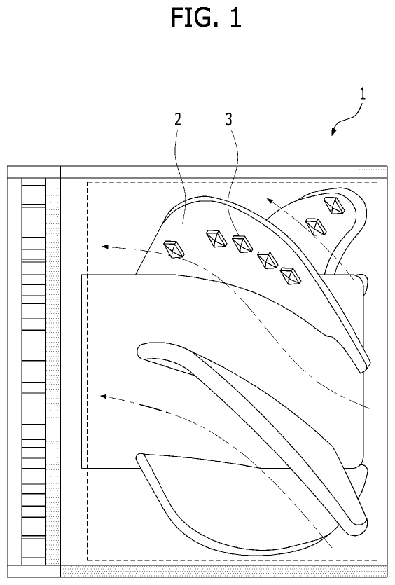

As illustrated in FIG. 1, Korean Patent No. 2024556 discloses a sterilization fan structure 1 comprising an ultraviolet light-emitting diode 3 in which the ultraviolet light-emitting diode 3 is coupled to a fan blade 2, which may not only decontaminate the air passing through the fan, but also eliminate microorganisms adhering to and living in the fan and contamination of the fan.

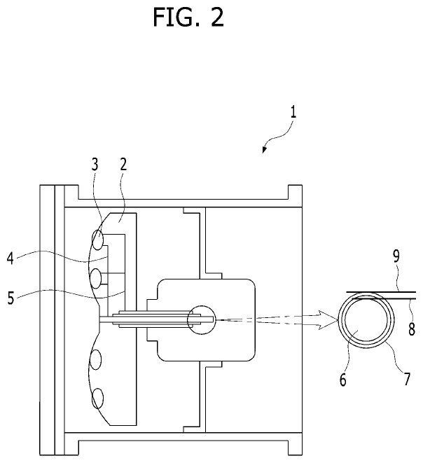

However, as illustrated in FIG. 2, in the sterilization fan structure of Korean Patent No. 2024556, an ultraviolet light-emitting diode 3 having a sterilization function is coupled to a blade 2 and rotates together with the blade. Therefore, in order to supply power to the rotating ultraviolet light-emitting diode 3, one end of a (+) wire 4 and one end of a (−) wire 5 need to be connected to the light-emitting diode 3, the other end of the (+) wire 4 and the other end of the (−) wire 5 need to be connected to (+)(−) electrode rods 6 and 7, and brushes 8 and 9 need to be connected to the electrode rods 6 and 7. In this structure, the electrode rods 6 and 7 and the brushes 8 and 9 are used to supply power to the ultraviolet light-emitting diode 3, which not only complicates the structure of the fan structure, but also degrades the efficiency of sterilization and the efficiency of an air conditioner, an air purifier or a vehicle air purification device due to defects in contact between the electrode rods and the brushes, and requires regular replacement of the brushes.

In an air blower 100 having a sterilization module integrated therewith according to the embodiment of the present invention, a sterilization module 40 is disposed on a stationary part 10 to radiate (emit) light (rays) upward, such that the sterilization module 40 may be maintained in a stationary state even though a rotary part 20 operates. Therefore, the electrode rod and the brush structure, which are configured to supply power to the sterilization module, are not used in the blower fan having the sterilization module integrated therewith, which may improve the efficiency of the sterilization operation of the blower fan, simplify the structure of the blower fan, and facilitate maintenance.

The air blower 100 having the sterilization module integrated therewith according to the embodiment of the present invention includes: the stationary part 10 including a central shaft 12 and a coil 18; the rotary part 20 including blades 26 and permanent magnets 28; a bearing 30 having an inner race fixed to the stationary part and an outer race fixed to the rotary part; and the sterilization module 40 disposed on the stationary part 10 so as to emit light upward.

The stationary part 10 of the air blower 100 may include the central shaft 12 protruding upward from a center thereof, a bracket 16 extending horizontally from a lower end of the central shaft, and the coil 18 disposed around the central shaft. The stationary part 10 is fixed to an air cleaner or vehicle air purification device and serves to support the rotary part 20 so that the rotary part 20 is rotatable. The bracket 16 disposed on a lower portion of the stationary part 10 is fixed to the air cleaner or vehicle air purification device (see FIGS. 6 and 7). In addition, a printed circuit board 51 may be disposed on an upper surface of the bracket 16, and a line (wire) for supplying power to the coil 18 may be connected to the printed circuit board 51. When power is supplied to the coil 18, the coil 18 serves as an electromagnet that generates an electromagnetic force.

The rotary part 20 of the air blower 100 is rotatably supported on the stationary part 10 so as to rotate about a central shaft of the stationary part 10, and the blades 26 are provided on a peripheral portion of the rotary part 20 (see FIGS. 5 and 7). The permanent magnets 28 are disposed inside the blades. The permanent magnets 28 are coaxially disposed while maintaining a predetermined gap with the coil 18 of the stationary part 10. As described above, the permanent magnets 28 of the rotary part are coaxially disposed around the coil 18 of the stationary part. Therefore, the permanent magnets 28 of the rotary part are rotated relative to the coil 18 of the stationary part by an interaction between an electromagnetic force, which is generated by an electric current applied to the coil 18 of the stationary part, and a magnetic force generated by the permanent magnets. The rotational motion allows the blades 26 provided on the rotary part to perform an air blowing operation.

The rotary part 20 and the stationary part 10 are coupled by means of the bearing 30 so that the rotary part 20 may smoothly rotate relative to the stationary part 10. In this case, an inner race 32 of the bearing 30 is fixed to the stationary part 10, and an outer race 34 of the bearing 30 is fixed to the rotary part 20. The bearing 30 may be configured as a rolling bearing. For example, the bearing 30 may be configured as a ball bearing or a roller bearing. In addition, the bearing 30 may also be configured as a sliding bearing.

According to a first embodiment of the present invention, as illustrated in FIG. 8, in the blower fan 100 having the sterilization module integrated therewith, a rotary shaft 22 of the rotary part 20 and the bearing 30 are disposed inside the coil 18 of the stationary part 10. In this case, it is possible to decrease a diameter of the bearing 30, which may reduce costs required to manufacture the bearing 30, implement the compact bearing 30, and improve durability of the bearing.

In the blower fan 100 having the sterilization module integrated therewith according to the first embodiment, the central shaft 12 of the stationary part 10 may include: a first protruding portion 13 extending from an upper surface of the bracket 16 to a predetermined height; and a connection part 14 fixedly inserted into the first protruding portion and extending to an upper surface of the rotary part 20. That is, one end of the connection part 14 is inserted into an upper end of the first protruding portion 13 extending from the upper surface of the bracket 16, and the other end of the connection part 14 extends to the upper surface of the rotary part 20.

In the first embodiment, the inner race 32 of the bearing 30 is fixed to an outer peripheral surface of the connection part 14 of the stationary part, and the outer race 34 of the bearing 30 is fixed to an inner peripheral surface of the rotary shaft 22 of the rotary part. As described above, because the inner race 32 of the bearing 30 is fixed to the connection part 14 of the stationary part having a comparatively small diameter, a diameter of the bearing 30 may be decreased.

In the first embodiment, the sterilization module 40 is disposed at an upper end of the connection part 14 of the stationary part 10 so as to be exposed outward, such that the sterilization module 40 may diffusely radiate light (rays) upward. Further, because the sterilization module 40 is disposed on the connection part 14 of the stationary part 10, the sterilization module 40 is maintained in a stationary state without being rotated together with the rotary part even when the rotary part 20 rotates. With this configuration, an electrode rod and a brush structure, which are configured to supply power to the sterilization module, are not used for the blower fan, such that the efficiency of the sterilization operation of the blower fan is improved, the structure of the blower fan is simplified, and the maintenance is easily performed.

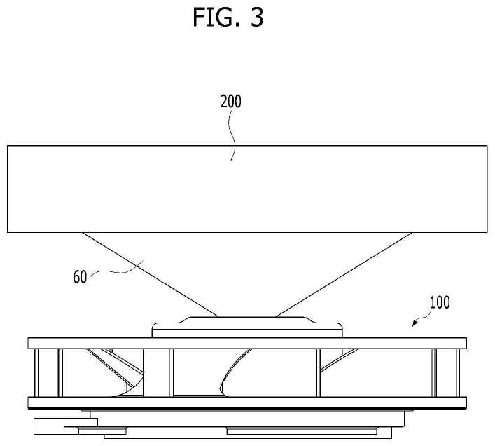

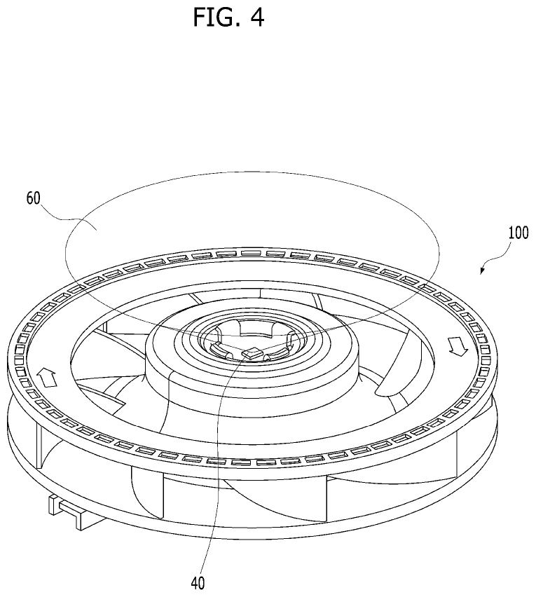

FIG. 4 illustrates that light is radiated and diffused upward from the sterilization module 40 of the blower fan 100 having the sterilization module 40 integrated therewith according to the first embodiment in the vehicle air purification device. As described above, the sterilization module 40 is disposed on the stationary part 10 of the blower fan, but the light is diffused upward and defines an irradiation region 60, which makes it possible to completely cover a sterilization target disposed above the vehicle air purification device. FIG. 3 schematically illustrates that a filter unit 200 is disposed above the blower fan 100 having the sterilization module 40 integrated therewith according to the first embodiment of the present invention in the vehicle air purification device. In case that the sterilization module 40 is disposed on the filter unit 200 as described above, a flow velocity of air passing through the filter unit 200 decreases, and an effect of sterilizing air is improved. Since the irradiation region 60, in which light is diffused upward, is formed, the entire filter unit 200 may be directly sterilized.

In the blower fan having the sterilization module integrated therewith according to the present invention, the sterilization module 40 may be configured as a UV-C LED.

The ultraviolet ray (UV) is light with a wavelength range of 10 to 400 nm and collectively referred to as an electromagnetic wave having a wavelength shorter than a wavelength of visible light and longer than a wavelength of an X-ray. UV rays may be classified by wavelength into UV-C with a wavelength of 100 to 280 nm, UV-B with a wavelength of 280 to 320 nm, and UV-A with a wavelength of 320 to 400 nm. These ultraviolet rays are chemically active and may destroy many molecules in a cell when the cell is exposed to strong ultraviolet radiation. In particular, ultraviolet rays with a wavelength of about 250 nm destroy DNA and are excellent for sterilization. Ultraviolet rays in this 250 nm wavelength band are UV-C. As such, UV rays are harmful to humans when the human body is directly irradiated because UV rays destroy molecules in cells, so caution is required when using UV rays.

When the blower fan 100 having the sterilization module 40 integrated therewith according to the present invention is applied to the vehicle air purification device, the blower fan 100 is disposed rearward of the filter unit in a housing of the vehicle air purification device. Therefore, the sterilization module is not exposed to the outside, and the UV-C LED may be safely used for the sterilization module 40 according to the present invention. Since UV-C has a shorter wavelength compared to UV-A or UV-B, the UV-C requires less energy but has a higher sterilization effect, so the UV-C may be used in air purifiers or vehicle air purification devices in which the sterilization module is not directly exposed to the outside.

Next, a blower fan having a sterilization module integrated therewith according to a second embodiment of the present invention will be described. The description of the parts identical or similar to those in the first embodiment will be omitted, and the parts different from those in the first embodiment will be described.

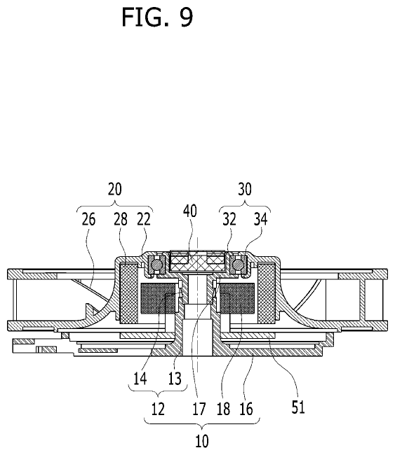

According to the second embodiment of the present invention, as illustrated in FIG. 9, in the blower fan 100 having the sterilization module integrated therewith, a rotary shaft 23 of the rotary part 20 and the bearing 30 are disposed inside the permanent magnets 28 of the rotary part 20. In this case, in comparison with the first embodiment in FIG. 8, a diameter of the bearing 30 may be increased, which makes it easy to perform a wiring process of connecting a power supply line to the sterilization module 40.

In the blower fan 100 having the sterilization module integrated therewith according to the second embodiment, the central shaft 12 of the stationary part 10 may include: the first protruding portion 13 extending from the upper surface of the bracket 16 to a predetermined height; and a connection part 15 fixedly inserted into the first protruding portion and having a flange portion 17 provided at an upper end thereof and extending horizontally.

In the second embodiment, the inner race 32 of the bearing 30 is fixed to the flange portion 17 of the connection part 15 of the stationary part, and the outer race 34 of the bearing 30 is fixed to an inner peripheral surface of the rotary shaft 23 of the rotary part. As described above, because the inner race 32 of the bearing 30 is fixed to the flange portion 17 of the connection part 15 of the stationary part having a comparatively large diameter, a diameter of the bearing 30 may be comparatively increased, which makes it easy to perform the wiring process of connecting the power supply line to the sterilization module 40.

Even in the second embodiment, as in the first embodiment, the sterilization module 40 is disposed on the stationary part 10, such that the sterilization module 40 is maintained in the stationary state without being rotated together with the rotary part even when the rotary part 20 rotates. With this configuration, an electrode rod and a brush structure, which are configured to supply power to the sterilization module, are not used for the blower fan, such that the efficiency of the sterilization operation of the blower fan is improved, the structure of the blower fan is simplified, and the maintenance is easily performed.

Next, a blower fan having a sterilization module integrated therewith according to a third embodiment of the present invention will be described. The description of the parts identical or similar to those in the first and second embodiments will be omitted, and the parts different from those in the first and second embodiments will be described.

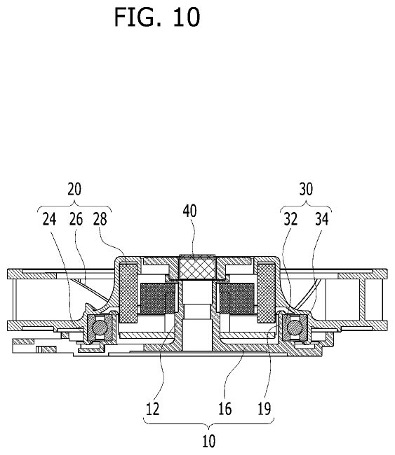

According to the third embodiment of the present invention, as illustrated in FIG. 10, in the blower fan 100 having the sterilization module integrated therewith, the rotary shaft 23 of the rotary part 20 and the bearing 30 are disposed outside the permanent magnets 28 of the rotary part 20. In this case, in comparison with the first embodiment in FIG. 8 and the second embodiment in FIG. 9, a diameter of the bearing 30 may be further increased, which makes it much easier to perform the wiring process of connecting the power supply line to the sterilization module 40.

In the blower fan 100 having the sterilization module integrated therewith according to the third embodiment, the stationary part 10 may further include a second protruding portion 19 disposed outside the first protruding portion 13 and extending from the upper surface of the bracket 16 to a predetermined height concentrically with the first protruding portion.

In the third embodiment, the inner race 32 of the bearing 30 is fixed to an outer peripheral surface of the second protruding portion 19 of the stationary part, and the outer race 34 of the bearing 30 is fixed to an inner peripheral surface of the rotary shaft 24 of the rotary part. As described above, since the inner race 32 of the bearing 30 is fixed to the second protruding portion 19 of the stationary part having a comparatively large diameter, a diameter of the bearing 30 may be increased, which makes it much easier to perform the wiring process of connecting the power supply line to the sterilization module 40.

Even in the third embodiment, as in the first and second embodiments, the sterilization module 40 is disposed on the stationary part, such that the sterilization module 40 is maintained in the stationary state without being rotated together with the rotary part even when the rotary part 20 rotates. With this configuration, an electrode rod and a brush structure, which are configured to supply power to the sterilization module, are not used for the blower fan, such that the efficiency of the sterilization operation of the blower fan is improved, the structure of the blower fan is simplified, and the maintenance is easily performed.

The example has been described above in which the embodiment of the present invention is applied to the vehicle air purification device. The blower fan having the sterilization module integrated therewith according to the embodiment of the present invention may be used for air conditioners or air cleaners used indoors, air conditioners or air conditioning systems used at industrial sites, and the like to sterilize filter units and used to sterilize cooling fans in vehicle engine rooms, evaporators of vehicle air conditioning systems, or the like.

While the present invention has been described with reference to the embodiment illustrated in the drawings, the embodiment is described just for illustration, and those skilled in the art to which the present invention pertains will understand that various modifications of the embodiment and any other embodiment equivalent thereto are available. Accordingly, the true technical protection scope of the present invention should be determined by the technical spirit of the appended claims.

1. A blower fan having a sterilization module integrated therewith, the blower fan comprising:
a stationary part 10 including a central shaft 12 protruding upward from a center thereof, a bracket 16 extending horizontally from a lower end of the central shaft, and a coil 18 disposed around the central shaft;
a rotary part 20 including a blade 26 formed on a peripheral portion thereof, and a permanent magnet 28 disposed around the coil of the stationary part at the inner side of the blade, the rotary part 20 being configured to rotate about the central shaft of the stationary part;
a bearing 30 having an inner race 32 fixed to the stationary part 10 and an outer race 34 fixed to the rotary part 20; and
a sterilization module 40 disposed at an upper end of the central shaft of the stationary part to diffusely radiate light upward, the sterilization module 40 being configured to be maintained in a stationary state when the rotary part 20 rotates.

2. The blower fan of claim 1, wherein the sterilization module 40 is configured as a UV-C LED.

3. The blower fan of claim 1, wherein the central shaft 12 of the stationary part comprises a first protruding portion 13 extending from an upper surface of the bracket 16 to a predetermined height and a connection part 14 fixedly inserted into the first protruding portion and extending to an upper surface of the rotary part,
wherein the inner race 32 of the bearing 30 is fixed to an outer peripheral surface of the connection part 14 of the stationary part, and the outer race 34 is fixed to an inner peripheral surface of a rotary shaft 22 of the rotary part, and
wherein the sterilization module 40 is disposed at the upper end of the connection part of the stationary part 10 to diffusely radiate light upward and maintained in the stationary state when the rotary part 20 rotates.

4. The blower fan of claim 1, wherein the central shaft 12 of the stationary part comprises a first protruding portion 13 extending from an upper surface of the bracket 16 to a predetermined height and a connection part 15 fixedly inserted into the first protruding portion and having a flange portion 17 provided at an upper end thereof and extending horizontally,
wherein the inner race 32 of the bearing 30 is fixed to a peripheral portion of the flange portion 17 of the connection part of the stationary part, and the outer race 34 is fixed to an inner peripheral surface of a rotary shaft 23 of the rotary part, and
wherein the sterilization module 40 is disposed on the flange portion of the connection part of the stationary part 10 to diffusely radiate light upward and maintained in the stationary state when the rotary part 20 rotates.

5. The blower fan of claim 1, wherein the stationary part 10 further comprises a second protruding portion 19 disposed outside a first protruding portion 13 and extending from an upper surface of the bracket 16 to a predetermined height concentrically with the first protruding portion,
wherein the inner race 32 of the bearing 30 is fixed to an outer peripheral surface of the second protruding portion 19 of the stationary part, and the outer race 34 is fixed to an inner peripheral surface of a rotary shaft 24 of the rotary part.

6. The blower fan of claim 1, wherein the bearing 30 is a ball bearing.

7. A vehicle air purification device comprising the blower fan 100 having a sterilization module integrated therewith according to claim 1.

8. The vehicle air purification device of claim 7, wherein the blower fan 100 having the sterilization module integrated therewith is disposed rearward of a filter unit 200.
